(19) United States
(12) Patent Application Publication
(10) Pub. No.: US 2004/0220275 A1
(43) Pub. Date: Nov. 4, 2004

(54) Fungicidal compositions of disinfecting and preserving agents

(76) Inventors: Michael Lutzeler, Grenzach-Wyhlen (DE); Florian Lichtenberg, Grenzach-Wyhlen (DE)

Correspondence Address: Fisher Christen & Sabol, Suite 1108, 1725 K Street NW, Washington, DC, 20006, US

(21) Appl. No.: 10/487,923

(22) PCT Filed: Aug. 22, 2002
(86) PCT No.: PCT/EP02/09383

Related U.S. Application Data
(60) Provisional application No. 60/401,808, filed on Aug. 8, 2002.

(30) Foreign Application Priority Data
Aug. 31, 2001 (EP) 01120978.0

(51) Int. Cl. A61K 31/14

(52) U.S. Cl. 514/642

(57) ABSTRACT

A fungicidal composition of disinfecting and preserving agents, including at least both an amine and/or quaternary ammonium salt of the general formula

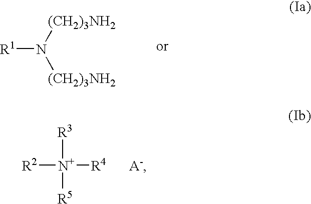

in which R¹ is C₆₋₈-alkyl, R² is benzyl or C₆₋₈-alkyl, R³ is C₁₋₁₈-alkyl or -[(CH ₂)₂-O]-R⁶ with n=1−20, R⁴ and R⁵ independently of one another are C₁₋₄-alkyl, R⁶ is hydrogen or optionally substituted phenyl and A is a monovalent anion or an equivalent of a polyvalent anion of an inorganic or organic acid, and also a sulfite or dithionite of the general formula M(HSO₃) (lla) and/or M₂S₂O₅(llb) and/or M₂S₂O₄ (llc) and/or M₂SO₃ (lld), in which M is a monovalent cation or an equivalent or a polyvalent cation, in the l:ll ration of from 20:1 to 1:20 by mass.

The invention relates to synergistic compositions of disinfecting and preserving agents based on amines and/or quaternary ammonium salts.

Amines and/or quaternary ammonium salts with alkyl and/or aralkyl side chains are used as active substances in numerous compositions of disinfecting and preserving agents. These include, for example, N,N-bis(3-aminopropyl)octylamine (WO-A-01/00024), N,N-bis(3-aminopropyl)dodecylamine (EP-A-0 333 143, EP-A-0 343 605) and single or multiple (WO-A-90/09405) ammonium salts. However, the corresponding compositions generally show, especially at higher dilutions, an unsatisfactory activity on fungi such as, for example, Aspergillus niger.

It was therefore an object of the present invention to provide compositions of disinfecting and preserving agents based on amines and/or quaternary ammonium salts which show, even at high dilution, a good activity on fungi, in particular not only in the alkaline region but also under neutral to acidic pH conditions.

This object is achieved according to the invention by the disinfectant composition as claimed in claim 1.

It has surprisingly been found that amines and or quaternary ammonium salts of the general formula

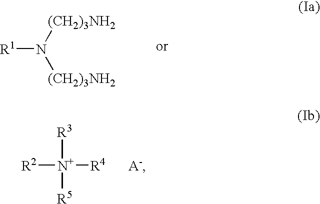

in which R¹ is C₆₋₁₈-alkyl,

R² is benzyl or C₆₋₁₈-alkyl,

R³ is C₁₋₁₈-alkyl or —[(CH₂)₂—O]ₙ—R⁶ with n=1-20,

R⁶ is hydrogen or optionally substituted phenyl and

A⁻ is a monovalent anion or an equivalent of a polyvalent anion of an inorganic or organic acid,

acquire good fungicidal activity through addition of at least one sulfite or dithionite of the general formula M(HSO₃) (IIa), M₂S₂O₅ (IIb), M₂S₂O₄ (IIc) or M₂SO₃ (IId), in which M is a monovalent cation or an equivalent of a polyvalent cation, in the ratio (I:II) of from 20:1 to 1:20 by mass.

Alkyl means here and hereinafter in each case linear or branched alkyl groups having the stated numbers of carbons, but preferably linear alkyl groups and particularly preferably those having an even number of carbon atoms. In particular, this also means the homolog mixtures derived from natural raw materials, such as, for example, “cocoalkyl”.

Substituted phenyl groups mean in particular phenyl groups substituted by one or more C₁₋₈-alkyl groups and/or chlorine atoms.

Suitable in principle as anion A⁻ are all inorganic or organic anions, in particular halides such as, for example, chloride or bromide or anions of lower carboxylic acids such as, for example, acetate, propionate, succinate, or lactate, and carbonate or methyl carbonate.

Suitable and preferred amines and quaternary ammonium salts (Ia/Ib) are

N,N-bis(3-aminopropyl)dodecylamine,

N,N-bis(3-aminopropyl)octylamine,

didecyldimethylammonium salts,

octyldecylammonium salts,

dioctyldimethylammonium salts,

dicocoalkyldimethylammonium salts,

cocoalkyldimethylpoly(oxyethyl)ammonium salts,

dicocoalkylmethylpoly(oxyethyl)ammonium salts,

decyldimethylpoly(oxyethyl)ammonium salts,

didecylmethylpoly(oxyethyl)ammonium salts,

octyldimethylpoly(oxyethyl)ammonium salts,

dioctylmethylpoly(oxyethyl)ammonium salts,

cocoalkylbenzyldimethylpoly(oxyethyl)ammonium salts,

cocoalkylbenzylethylmethylpoly(oxyethyl) ammonium salts,

benzyldodecyldimethylammonium salts or

benzyldimethylpoly(oxyethyl)ammonium salts or

any mixtures of at least two of these compounds.

Suitable sulfites and dithionites of the general formulae IIa-d are all soluble bisulfites (MHSO₃, IIa), disulfites (M₂S₂O₅, IIb), dithionites (M₂S₂O₄, IIc) or sulfites (M₂SO₃, IId) in which M is a monovalent cation or an equivalent of a polyvalent cation.

Particularly suitable cations M of the sulfites or dithionites (II) are all alkali metal, alkaline earth metal, ammonium, copper and silver ions, preferably sodium, potassium and ammonium ions.

It is, of course, also within the scope of the invention to employ mixtures of said compounds.

Particularly good results are obtained with sulfites (IId), especially with sodium sulfite.

The ratio of amine (Ia) or quaternary ammonium salt (Ib) to the sulfites or dithionites (IIa-d) used is preferably in the range from 1:5 to 5:1 by mass.

The preferred solvent for the novel compositions of disinfecting and preserving agents is water, where appropriate combined with an organic solvent preferably of limited or unlimited miscibility with water.

The novel compositions of disinfecting and preserving agents preferably also comprise one or more auxiliaries from the group consisting of organic solvents, surfactants, buffers, complexing agents, fragrances and dyes.

A further aspect of the invention relates to the use of sodium thiosulfate as stabilizer for the novel compositions of disinfecting and preserving agents. It is known that the pH of sodium sulfite solutions decreases after only a short time through elimination inter alia of SO₂. The synergistic effect, described hereinbefore, of sulfite on the fungicidal and bactericidal effect of amines and ammonium salts is abolished again after a few days through the degradation of the added sulfite. In addition, the reduction in pH then brings about a loss of effect of the amines and ammonium salts used. In order to be able to stabilize the pH of the solutions long-term, and thus achieve a commercially utilizable storage period, various conventional buffer systems have been added. Conventional citrate-, acetate-, phosphate- or ammonium chloride-based buffer systems have proved to be ineffective in this connection.

However, it was possible to establish, surprisingly, that the use of thiosulfate (for example sodium thiosulfate, Na₂S₂O₃) brings about an exceptional stabilization of the pH compared with all other buffers used. No reduction in pH was detectable even 21 days after making up the solutions. The stabilizing effect was also confirmed after admixing the amines and ammonium salts.

An increase in the buffering effect, which was itself very good, of sodium thiosulfate was observable in combination with sodium dihydrogen phosphate (NaH₂PO₄.H₂O).

A preferred area of use of the novel compositions of disinfecting and preserving agents is the disinfection of surfaces and instruments.

Further preferred areas of use are the disinfection of laundry and hands.

The novel compositions of disinfecting and preserving agents are also very suitable for use in chemical (temporary) toilets such as, for example, on board aircraft and vehicles or on building sites.

A further preferred area of use is the preservation of industrial aqueous liquids such as, for example, water circulations in paper manufacturing, cooling water systems, belt lubricants for conveyor belts or cooling lubricants in metal processing, called metal working fluids.

A likewise preferred application is the use as protectant and preservative for organic and/or biologically vulnerable construction materials such as, for example, wood.

The following examples illustrate the implementation of the invention, but no restriction to the described embodiments is to be seen therein. It is also possible on the basis of the claimed variations of the individual components to formulate numerous further examples of the invention. These examples, which are made by alteration of the composition within the variations defined in the description and in the claims, are to be regarded as examples of the invention and fall within the scope of the protection of this patent application.

All stated amounts are, unless stated otherwise, stated in % by mass. The test microbe employed in each case was Aspergillus niger ATCC 16404. The activity was, unless otherwise indicated, determined by the method specified in CEN 1275.

### EXAMPLE 1

A disinfectant cleaner formulation (concentrate) was prepared from:

9.0% didecyldimethylammonium chloride (50% strength solution)

6.0% Genapol UD 080 (C₁₁ oxo alcohol, 8 mol of ethylene oxide)

6.0% butyl diglycol

4.5% sodium sulfite (Na₂SO₃)

1.0% sodium thiosulfate

0.3% sodium dihydrogen phosphate (NaH₂PO₄.H₂O)

73.2% water

pH of the concentrate: pH 7.9

The activity was determined with a dilution (2 parts of concentrate, 98 parts of water) at 20° C. with a contact time of 15 min. The decadic logarithm of the germ count reduction was 4.2.

### COMPARATIVE EXAMPLE 1

The procedure was as in Example 1 but with the difference that the sodium sulfite was replaced by the same amount of water. Under the same test conditions, the formulation was virtually inactive.

### EXAMPLE 2

A disinfectant cleaner formulation (concentrate) was prepared from:

9.9% N,N-bis(3-aminopropyl)dodecylamine

12.0% didecylmethylpoly(oxyethyl)ammonium propionate (BARDAP 26)

20.0% 1,4-butanediol

5.0% butyl diglycol

4.0% sodium sulfite (Na₂SO₃)

1.0% sodium thiosulfate

3.0% ethylenediaminetetraacetic acid

4.0% Genapol® T 250 (tallow fatty alcohol polyglycol ether, 25 mol of ethylene oxide)

2.0% Glucopon® 215 CSUP (C₈₋₁₀ fatty alcohol polyglycoside)

39.1% water

pH of the concentrate: pH 9.7

The activity was determined with a dilution (2 parts of concentrate, 98 parts of water) at 20° C. with a contact time of 15 min. The decadic logarithm of the germ count reduction was 4.2.

In addition the activity was also determined by the method specified in CEN 1650 with a contact time of 15 min, a concentration of 5.0%, a water hardness of 30° fH and an organic load of 0.3% albumin. The logarithm of the germ count reduction was >4.6.

### COMPARATIVE EXAMPLE 2

The procedure was as in Example 2 but with the difference that the sodium sulfite was replaced by the same amount of water. Under the same test conditions, the formulation was virtually inactive.

### EXAMPLES 3 to 18

Aqueous solutions were prepared from 0.5% sulfite or dithionite (II) and 0.25% amine or quaternary ammonium salt (Ia/Ib) and tested by the method specified in CEN 1275. The results are compiled in Table 1.

TABLE 1

| Ex- | | | lg germ |
|---|---|---|---|
| ample | | Sulfite/ | reduc- |
| No. | Amine/ammonium salt | dithionite | tion |
| 1 | didecyldimethylammonium | sodium sulfite | 4.2 |
| | chloride | (Na₂SO₃) | |
| C1 | didecyldimethylammonium | — | inactive |
| | chloride | | |
| 2 | N,N-bis(3-aminopropyl)- | Na₂SO₃ | 4.2 |
| | dodecylamine and | | |
| | didecylmethylpoly- | | |
| | (oxyethyl)ammonium | | |
| | propionate (BARDAP ™ 26) | | |
| C2 | N,N-bis(3-aminopropyl)- | — | inactive |
| | dodecylamine and | | |
| | didecylmethylpoly- | | |
| | (oxyethyl)ammonium | | |
| | propionate (BARDAP ™ 26) | | |
| 3 | dimethyldioctylammonium | Na₂SO₃ | 4.4 |
| | chloride | | |
| 4 | dimethyldioctylammonium | sodium | 4.2 |
| | chloride | dithionite | |
| | | (Na₂S₂O₄) | |
| 5 | dimethyldioctylammonium | sodium bisulfite | 3.1 |
| | chloride | (NaHSO₃) | |
| 6 | dimethyldioctylammonium | sodium disulfite | 4.1 |
| | chloride | (Na₂S₂O₅) | |
| 7 | didecyldimethylammonium | Na₂SO₃ | 4.2 |
| | chloride | | |
| 8 | didecyldimethylammonium | Na₂S₂O₄ | 4.0 |
| | chloride | | |
| 9 | didecyldimethylammonium | NaHSO₃ | 2.7 |
| | chloride | | |
| 10 | didecyldimethylammonium | Na₂S₂O₅ | 3.8 |
| | chloride | | |
| 11 | di-C₈₋₁₀-alkyldimethyl- | Na₂SO₃ | 4.0 |
| | ammonium chloride (60%)/ | | |
| | C₁₂₋₁₆-alkylbenzyl- | | |
| | dimethylammonium | | |
| | chloride (40%); Bardac ® | | |
| | 205-M | | |
| 12 | di-C₈₋₁₀-alkyldimethyl- | Na₂S₂O₄ | 3.7 |
| | ammonium chloride (60%)/ | | |
| | C₁₂₋₁₆-alkylbenzyl- | | |
| | dimethylammonium | | |
| | chloride (40%); Bardac ® | | |
| | 205-M | | |
| 13 | di-C₈₋₁₀-alkyldimethyl- | NaHSO₃ | 2.5 |
| | ammonium chloride (60%)/ | | |
| | C₁₂₋₁₆-alkylbenzyl- | | |
| | dimethylammonium | | |
| | chloride (40%); Bardac ® | | |
| | 205-M | | |
| 14 | di-C₈₋₁₀-alkyldimethyl- | Na₂S₂O₅ | 3.5 |
| | ammonium chloride (60%)/ | | |
| | C₁₂₋₁₆-alkylbenzyl- | | |
| | dimethylammonium | | |
| | chloride (40%); Bardac ® | | |
| | 205-M | | |
| 15 | N,N-bis(3-aminopropyl)- | Na₂SO₃ | 4.2 |
| | dodecylamine | | |
| 16 | N,N-bis(3-aminopropyl)- | Na₂S₂O₄ | 3.8 |
| | dodecylamine | | |
| 17 | N,N-bis(3-aminopropyl)- | NaHSO₃ | 2.4 |
| | dodecylamine | | |
| 18 | N,N-bis(3-aminopropyl)- | Na₂S₂O₅ | 3.9 |
| | dodecylamine | | |

For comparison, all the substances listed in Table 1 were tested as single substances in 0.5% strength solution. None of these compounds showed a marked fungicidal effect (lg germ reduction <2).

### EXAMPLE 19

Stabilizing Effect of Thiosulfate

To demonstrate the stabilizing effect of thiosulfate, where appropriate combined with dihydrogen phosphate, on sulfite-containing solutions, various buffer compositions were prepared and the pH was determined over the course of about 3 weeks (on days 1, 2, 5 and 22). The results are compiled in Table 2 below. The pH of the composition according to Example 1 was determined after one year.

TABLE 2

| | pH | | | | |
|---|---|---|---|---|---|
| Buffer compositions | day | day | day | day | 1 |
| (water in each case ad 100%) | 1 | 2 | 5 | 22 | year |
| 4.5% sodium sulfite (Na₂SO₃) | 10.2 | 9.9 | 9.3 | n.d. | n.d. |
| 4.5% Na₂SO₃ | 8.0 | 7.9 | 7.7 | n.d. | n.d. |
| 2.0% ammonium chloride | | | | | |
| 4.5% Na₂SO₃ | 7.7 | 7.6 | 7.4 | n.d. | n.d. |
| 0.3% NaH₂PO₄.H₂O | | | | | |
| 4.5% Na₂SO₃ | 7.6 | 7.6 | 7.6 | 7.6 | n.d. |
| 0.3% NaH₂PO₄.H₂O | | | | | |
| 1.0% sodium thiosulfate | | | | | |
| 9.0% didecyldimethylammonium | 7.9 | 7.9 | 7.9 | 7.9 | 7.8 |
| chloride (50% strength | | | | | |
| solution) | | | | | |
| 6.0% Genapol UD 080 (C₁₁ oxo | | | | | |
| alcohol, 8 mol of | | | | | |
| ethylene oxide) | | | | | |
| 6.0% butyl diglycol | | | | | |
| 4.5% Na₂SO₃ | | | | | |
| 1.0% sodium thiosulfate | | | | | |
| 0.3% NaH₂PO₄.H₂O | | | | | |

n.d. = not determined

1. A fungicidal composition of disinfecting and preserving agents, comprising at least
a) an amine and/or quaternary ammonium salt of the general formula 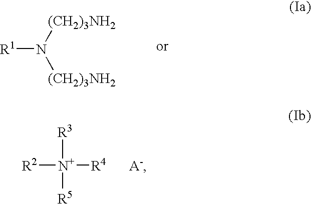
in which R¹ is C₆₋₁₈-alkyl,
R² is benzyl or C₆₋₁₈-alkyl,
R³ is C₁₋₁₈-alkyl or —[(CH₂)₂—O]ₙ—R⁶ with n=1-20,
R⁴ and R⁵ independently of one another are C₁₋₄-alkyl,
R⁶ is hydrogen or optionally substituted phenyl and
A⁻ is a monovalent anion or an equivalent of a polyvalent anion of an inorganic or organic acid, and
b) a sulfite or dithionite of the general formulae
M(HSO₃) (IIa) and/or M₂S₂O₅ (IIb) and/or M₂S₂O₄ (IIc) and/or M₂SO3 (IId),
in which M is a monovalent cation or an equivalent of a polyvalent cation,
in the I:II ratio of from 20:1 to 1:20 by mass.

2. A fungicidal composition of disinfecting and preserving agents as claimed in claim 1 characterized in that the amine or the quaternary ammonium salt of the general formula Ia or Ib is selected from the group consisting of
N,N-bis(3-aminopropyl)dodecylamine,
N,N-bis(3-aminopropyl)octylamine,
didecyldimethylammonium salts,
dioctyldimethylammonium salts,
octyldecyldimethylammonium salts,
dicocoalkyldimethylammonium salts,
cocoalkyldimethylpoly(oxyethyl)ammonium salts,
dicocoalkylmethylpoly(oxyethyl)ammonium salts,
decyldimethylpoly(oxyethyl)ammonium salts,
didecylmethylpoly(oxyethyl)ammonium salts,
octyldimethylpoly(oxyethyl)ammonium salts,
dioctylmethylpoly(oxyethyl)ammonium salts,
cocoalkyldimethylbenzylammonium salts,
cocoalkyldimethylethylbenzylammonium salts,
benzyldodecyldimethylammonium salts,
benzyldimethylpoly(oxyethyl)ammonium salts and
mixtures of at least two of these compounds.

3. A fungicidal composition of disinfecting and preserving agents as claimed in claim 1, characterized in that M is an alkali metal, ammonium, copper or silver ion or an equivalent of an alkaline earth metal ion.

4. A fungicidal composition of disinfecting and preserving agents as claimed in claim 1, characterized in that the ratio of amine and/or quaternary ammonium salt to sulfite and/or dithionite is between 1:5 and 5:1 by mass.

5. A fungicidal composition of disinfecting and preserving agents as claimed in claim 1, characterized in that it comprises water as solvent.

6. A fungicidal composition of disinfecting and preserving agents as claimed in claim 1, characterized in that it additionally comprises one or more auxiliaries from the group consisting of organic solvents, surfactants, buffers, complexing agents, fragrances and dyes.

7. A fungicidal composition of disinfecting and preserving agents as claimed in claim 1, characterized in that it additionally comprises a thiosulfate.

8. A fungicidal composition of disinfecting and preserving agents as claimed in claim 7, characterized in that it additionally comprises an alkali metal dihydrogen phosphate.

9. The use of the fungicidal compositions of disinfecting and preserving agents as claimed in claim 1 for disinfecting surfaces and instruments.

10. The use of the fungicidal compositions of disinfecting and preserving agents as claimed in claim 1 for disinfecting laundry.

11. The use of the fungicidal compositions of disinfecting and preserving agents as claimed in claim 1 for disinfecting hands.

12. The use of the fungicidal compositions of disinfecting and preserving agents as claimed in claim 1 in chemical toilets.

13. The use of the compositions of disinfecting and preserving agents as claimed in claim 1 as preservative for industrial liquids.

14. The use of the compositions of disinfecting and preserving agents as claimed in claim 1 as preservative for belt lubricants for conveyor belts, cooling lubricants in metal processing and/or other metal working fluids.

15. The use of the compositions of disinfecting and preserving agents as claimed in claim 1 as protectant and preservative for construction materials.

16. The use of the compositions of disinfecting and preserving agents as claimed in claim 1 as protectant and preservative for wood.

17. A fungicidal composition of disinfecting and preserving agents as claimed in claim 2, characterized in that M is an alkali metal, ammonium, copper or silver ion or an equivalent of an alkaline earth metal ion.

18. A fungicidal composition of disinfecting and preserving agents as claimed in claim 17, characterized in that the ratio of amine and/or quaternary ammonium salt to sulfite and/or dithionite is between 1:5 and 5:1 by mass.

19. A fungicidal composition of disinfecting and preserving agents as claimed in claim 18, characterized in that it comprises water as solvent.

20. A fungicidal composition of disinfecting and preserving agents as claimed in claim 19, characterized in that it additionally comprises one or more auxiliaries from the group consisting of organic solvents, surfactants, buffers, complexing agents, fragrances and dyes.

21. A fungicidal composition of disinfecting and preserving agents as claimed in claim 20, characterized in that it additionally comprises a thiosulfate.

22. The use of the fungicidal compositions of disinfecting and preserving agents as claimed in claim 8 for disinfecting surfaces and instruments.

23. The use of the fungicidal compositions of disinfecting and preserving agents as claimed in claim 8 for disinfecting laundry.

24. The use of the fungicidal compositions of disinfecting and preserving agents as claimed in claim 8 for disinfecting hands.

25. The use of the fungicidal compositions of disinfecting and preserving agents as claimed in claim 8 in chemical toilets.

26. The use of the compositions of disinfecting and preserving agents as claimed in claim 8 as preservative for industrial liquids.

27. The use of the compositions of disinfecting and preserving agents as claimed in claim 8 as preservative for belt lubricants for conveyor belts, cooling lubricants in metal processing and/or other metal working fluids.

28. The use of the compositions of disinfecting and preserving agents as claimed in claim 8 as protectant and preservative for construction materials.

29. The use of the compositions of disinfecting and preserving agents as claimed in claim 8 as protectant and preservative for wood.
